(19) United States
(12) Patent Application Publication
(10) Pub. No.: US 2009/0318875 A1
(43) Pub. Date: Dec. 24, 2009

(54) DEVICES AND METHODS TO TREAT NASAL PASSAGES

(75) Inventor: Oren Friedman, Rochester, MN (US)

Correspondence Address: FISH & RICHARDSON P.C., PO BOX 1022, MINNEAPOLIS, MN, 55440-1022, US

(73) Assignee: MAYO FOUNDATION FOR MEDICAL EDUCATION AND RESEARCH, Rochester, MN (US)

(21) Appl. No.: 12/300,743

(22) PCT Filed: May 7, 2007
(86) PCT No.: PCT/US2007/068370

§ 371 Date: Mar. 6, 2009

Related U.S. Application Data
(60) Provisional application No. 60/800,573, filed on May 15, 2006.

(51) Int. Cl. A61M 5/31; A61F 2/18

(52) U.S. Cl. 604/187; 606/99

(57) ABSTRACT

Some embodiments of a nasal implant delivery system may provide minimally invasive insertion of one or more implants in suitable tissue planes of the nasal rim, the lateral nasal wall, or both. Such a delivery system may be minimally invasive in that it does not require a surgical intervention or an incision that cuts a portion of the nasal tissue. Rather, in some embodiments, the nasal implant delivery system may include a needle or other cannula device having a curvature at the distal portion adapted to penetrate into a targeted tissue plane in the nasal rim, the lateral nasal wall, the nasal septum, or the columella. Also, some embodiments of a delivery system can be used to apply a nasal rigidity supplement to internal or external portions of the nasal rim, the lateral nasal wall, or other nasal structures.

### CROSS-REFERENCE TO RELATED APPLICATION

This application claims priority to U.S. Patent Application No. 60/800,573 filed on May 15, 2006 and entitled “Delivery of Nasal Implants,” the entire contents of which is incorporated herein by reference.

### TECHNICAL FIELD

This document relates to delivering materials or devices for treatment of nasal tissue.

### BACKGROUND

Upper airway obstruction (e.g., snoring, sleep apnea, nasal breathing disorders, and the like) may occur as a result of obstructive anatomic elements located in the oral cavity, oropharynx, nasal cavity, nasopharynx, hypopharynx, or larynx. Common sites of obstruction include the tongue, the soft palate, and the nasal cavity. Snoring may result from vibrations of any one of a number of surfaces or structures of the pharynx, such as the soft palate, tongue base, or pharyngeal walls. Sleep apnea may result from partial or complete collapse of the upper airway and pharyngeal walls during sleep. Both snoring and obstructive sleep apnea may be exacerbated by a variety of anatomical findings in the nasal cavity.

Upper airway obstruction has been treated with a number of different therapies, such as the implantation of stiffening devices into the soft palate to improve airflow through the pharynx. These stiffening implants may act to change airflow patterns and alleviate some airway obstructions. Other techniques have focused on the tongue base in an effort to stiffen it or to reduce its size. A multitude of surgical techniques are available for the treatment of nasal obstruction, and there are a limited few minimally invasive techniques for the treatment of nasal obstruction.

Nasal obstruction most commonly occurs in isolation, but may also be associated with snoring and sleep apnea. The vast majority of techniques available for the treatment of nasal obstruction include surgical procedures in which a scalpel is used to incise the patient's nasal tissue for access to the nose. Subsequently, strengthening techniques through tissue rearrangement and suture reinforcement may be used to improve a patient's nasal airflow. Additional techniques are available in which an incision is made and implants of various materials are placed to help strengthen the walls of the nose. After a nasal implant is inserted, the surgical incision may be closed in a standard fashion. Many techniques for improving nasal obstruction involve surgical intervention in the form of septoplasty, septorhinoplasty, nasal reconstruction, nasal vestibular stenosis repair, nasal valve collapse repair, or other surgeries which require incisions. Surgically placed implants may fail when the implant is improperly positioned or if the body rejects the material. For example, when the implant device is not placed in a suitable tissue plane, the implant may either extrude through the mucosal membrane surface (functional failure) or be visible on the dermal surface (cosmetic failure).

### SUMMARY

Some embodiments of a nasal implant delivery system may provide minimally invasive insertion of one or more implants in suitable tissue planes of the nasal rim, the lateral nasal wall, the nasal septum, the columella, or all of these areas. Such a delivery system may be minimally invasive in that it does not require a surgical intervention or a scalpel incision that cuts a portion of the nasal tissue. Rather, in some embodiments, the nasal implant delivery system may include a needle or other cannula device having a curvature at the distal portion adapted to penetrate into a targeted tissue plane in the nasal rim, the lateral nasal wall, the nasal septum, or the columella.

In some embodiments, a nasal implant delivery system may include a delivery cannula having a distal portion, a proximal portion, and a lumen extending therebetween. The delivery system may also include a handle member coupled with the delivery cannula. The handle member may extend at least partially downward from the proximal portion of delivery cannula. The delivery system may also include an actuation member movably disposed in the lumen. The actuation member can include a distal surface to at least partially engage an implant device when the implant device is disposed in the lumen. The distal portion of the delivery cannula may include a curved section extending at least partially upward and generally away from the handle member that extends at least partially downward.

In one aspect, the curved section of the distal portion of the delivery cannula may be penetrable into a tissue plane of a nasal rim. For example, the curved section of the delivery cannula may extend in a generally arcuate shape toward the upward direction.

In another aspect, the curved section of the distal portion of the delivery cannula may be penetrable into a tissue plane of a lateral nasal wall. For example, the curved section of the delivery cannula may include a first curve that extends at least partially upward and a second curve that extends in a generally longitudinal direction toward a distal tip of the delivery cannula.

In particular embodiments, a nasal implant delivery system may include a delivery cannula including a distal portion, a proximal portion, and a lumen extending therebetween. At least a section of the proximal portion may extend in longitudinal direction along a central axis. The distal portion of the delivery cannula may include a curved section that extends in an at least partially lateral direction away from the central axis and may include a port to deliver a nasal implant. The system may further include an actuation member movable disposed in the lumen. The actuation member may include a distal surface to at least partially engage the nasal implant when the implant is disposed in the lumen. Also, the system may include a handle member coupled with the delivery cannula to manipulate the position of the distal portion relative to a targeted site. The handle member may be coupled to a hand-adjustable switch to cause the actuation member to move in the lumen.

In other embodiments, a nasal implant delivery system may include a delivery cannula having a distal portion, a proximal portion, and at least one lumen extending therebetween. At least a section of the proximal portion may extend in longitudinal direction along a central axis. The distal portion of the delivery cannula may include a curved section that extends in an at least partially lateral direction away from the central axis and may also include a port in communication with the at least one lumen to deploy a nasal implant material. The system may further include a fluid reservoir in fluid communication with the at least one lumen. The fluid reservoir may contain a polymer material in a generally non-rigid state. The system may also include an actuation member to deploy the polymer material from the distal portion of the delivery cannula and to a targeted nasal tissue plane. The polymer material can transition to a resilient state when deployed from the distal portion.

In some embodiments, a delivery system for a nasal rigidity supplement may include a delivery instrument having a distal portion and a proximal portion. The proximal portion may extend in longitudinal direction along a central axis, and the distal portion of the delivery instrument may include an applicator to contact a targeted nasal tissue site. The delivery system may also include a rigidity supplement material arranged on the applicator of the delivery instrument. The rigidity supplement material may be in a generally non-rigid state when arranged on the applicator, and the rigidity supplement material can transition to a resilient state when deployed from the applicator to the targeted nasal tissue site.

These and other embodiments may provide one or more of the following advantages. First, the delivery system may be used without a surgical incision that cuts a portion of the nasal tissue, thereby providing minimally invasive insertion of one or more implants in suitable tissue planes of the nasal rim, the lateral nasal wall, the nasal septum, the columella, or all of these areas. Second, the delivery system may deliver implants to the nasal rim, the lateral nasal wall, the septum, or the columella to treat conditions or defects in the nasal anatomy, which may not be adequately treated by procedures on the soft palate. Third, the delivery system may include a distal tip portion that is curved in a manner to provide ready access to the nasal rim or the lateral nasal wall, thereby permitting a practitioner to operate the delivery system from an effective and efficient position relative to the patient. Fourth, the distal tip portion of the delivery system may access the nasal anatomy along an insertion path that positions the implant in a targeted tissue plane, which can reduce the likelihood of functional failure (e.g., the implant extruding through the mucosal membrane surface) and cosmetic failure (e.g., the implant being visible on the dermal surface). Fifth, implant device may comprise a biocompatible material (e.g., Dacron or the like) that is flexible enough to pass through the curved section of the delivery system and is rigid enough to at least partially stiffen the surrounding nasal anatomy, which can change the nasal airflow patterns and can alleviate certain airway obstructions.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a perspective view of a portion of a delivery system and implant devices disposed in nasal rims, in accordance with some embodiments.

FIG. 2 is a perspective view of the implant devices of FIG. 1 when in a non-flexed condition.

FIG. 3 is a perspective view of a portion of a delivery system and implant devices disposed in a nasal lateral wall, in accordance with another embodiment.

FIG. 4 is a perspective view of the implant devices of FIG. 4 when in a non-flexed condition.

FIG. 5 is a side view of a delivery system for implanting nasal implants, in accordance with some embodiments.

FIG. 6 is a cross-sectional view of an embodiment of a tip of the delivery system of FIG. 5.

FIG. 7 is a cross-sectional view of another embodiment of a tip of the delivery system of FIG. 5.

FIG. 8 is a perspective view of a portion of the delivery system of FIG. 5 penetrating into a nasal rim, in accordance with some embodiments.

FIG. 9 is a cross-sectional view of the nasal implant disposed in the nasal rim of FIG. 8.

FIG. 10 is another cross-sectional view of the nasal implant of FIG. 9.

FIG. 11 is a side view of a delivery system for implanting nasal implants, in accordance with some embodiments.

FIG. 12 is a cross-sectional view of an embodiment of a tip of the delivery system of FIG. 11.

FIG. 13 is a cross-sectional view of another embodiment of a tip of the delivery system of FIG. 11.

FIG. 14 is a perspective view of a portion of the delivery system of FIG. 11 penetrating into a nasal lateral wall, in accordance with some embodiments.

FIG. 15 is a cross-sectional view of the nasal implant disposed in the nasal rim of FIG. 14.

FIG. 16 is another cross-sectional view of the nasal implant of FIG. 15.

FIG. 17 is a perspective view of a delivery system for implanting nasal implants, in accordance with some embodiments.

FIG. 18 is a top view of the delivery system of FIG. 17.

FIG. 19 a perspective view of a portion of a delivery system and implants disposed in the nasal rims, in accordance with some embodiments.

FIG. 20 is a cross-sectional view of an embodiment of a tip of a delivery system, in accordance with some embodiments.

FIG. 21 is a perspective view of a portion of a delivery system and implants disposed in a nasal lateral wall, in accordance with another embodiment.

FIG. 22 a perspective view of a portion of a delivery system and nasal rigidity supplements disposed on the nasal rims, in accordance with some embodiments.

FIG. 23 is a perspective view of a portion of a delivery system and nasal rigidity supplements disposed on the nasal lateral walls, in accordance with another embodiment.

Like reference symbols in the various drawings indicate like elements.

### DETAILED DESCRIPTION OF ILLUSTRATIVE EMBODIMENTS

A nasal implant delivery system can provide nonsurgical insertion path for one or more implants in targeted tissue planes of the nasal rim, the lateral nasal wall, or both. In some embodiments, the nasal implant delivery system may include a needle or other cannula device having a curvature at the distal portion adapted to penetrate into the targeted tissue plane in the nasal rim or in the nasal lateral wall. The nasal implants delivered into the nasal tissue may be used to treat conditions such as snoring, sleep apnea, nasal breathing disorders, and the like. For example, weakened cartilaginous support in the nose (e.g., septum, upper lateral wall, columella, or other part of the nasal anatomy) can cause nasal obstruction and altered nasal shape. The nasal implants described herein may be disposed in the nasal rim of each nostril, the lateral walls on each side of the nose, or both so as to at least partially support the surrounding nasal tissue and change the nasal airflow patterns. Moreover, the delivery system described herein can position the implant devices in a targeted tissue plane to reduce the likelihood of functional failure (e.g., extruding through the mucosal membrane surface) and cosmetic failure (e.g., be visible on the dermal surface).

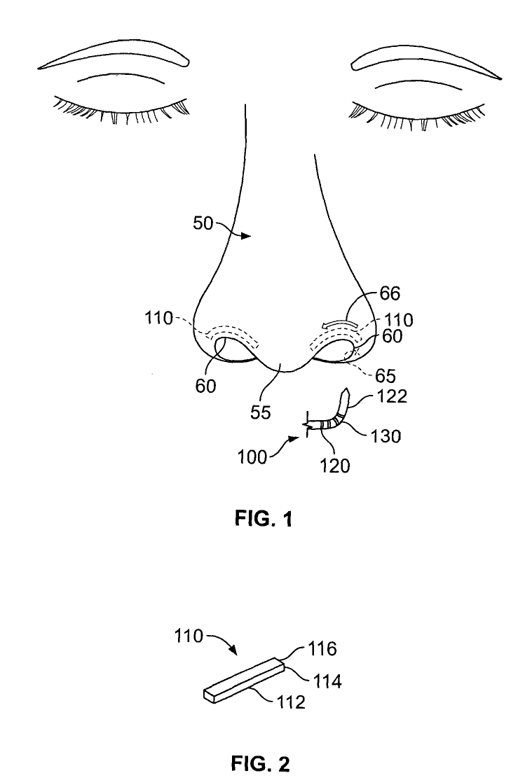

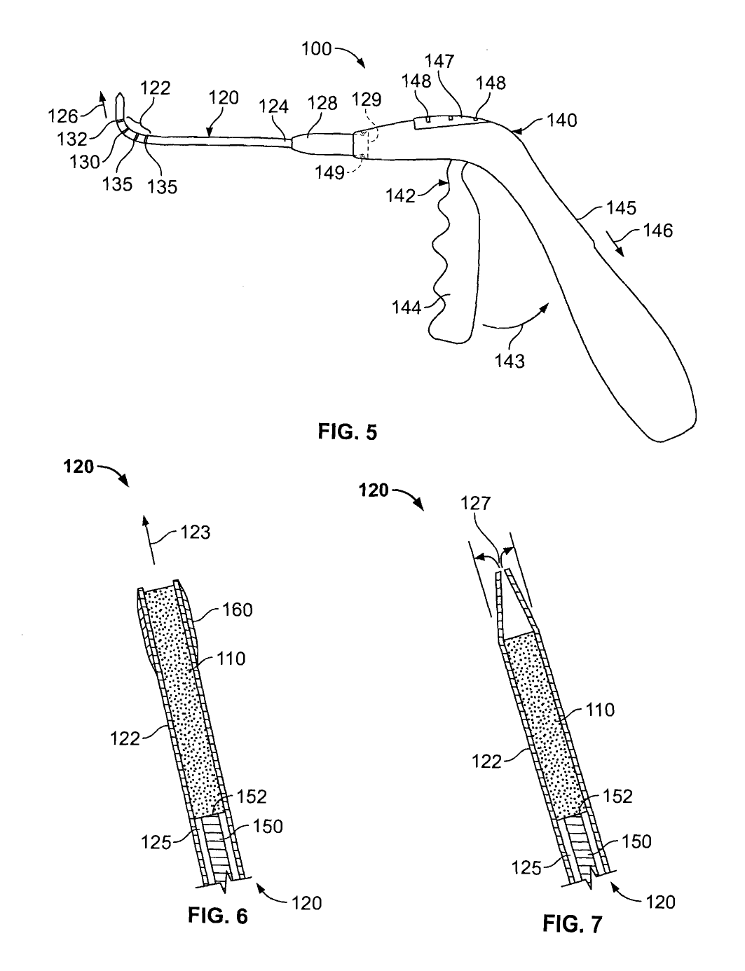

Referring to FIG. 1, a delivery system 100 may be inserted into nasal tissue proximate to the nostril rim 60 of a nose 50 to position a nasal implant device 110 therein. The delivery system 100 may include a delivery needle 120 or other cannula device (e.g., a specially configured hypotube, a polymer cannula, or the like) having sufficient rigidity to penetrate into the nasal tissue. The delivery needle 120 includes a distal tip portion 122, a proximal portion 124 (not shown in FIG. 1), and a lumen 125 extending therebetween (not shown in FIG. 1). As described in more detail below in connection with FIG. 5, the delivery system 100 may also include a handle member at a proximal portion coupled with the delivery needle 120. Also described in more detail below, an actuation member can be movably adjusted in the lumen of the delivery needle 120 to force the implant device 110 out from the distal tip portion 122 when the distal tip portion 122 is disposed in a targeted tissue plane of the nasal rim 60.

The distal tip portion 122 of the delivery needle 120 may include a curved section 130 extending at least partially in a lateral direction (e.g., a generally upward direction as shown, for example, in FIG. 5). Such a curved orientation may facilitate delivery of the implant device 110 into the targeted tissue plane of the nasal rim 60. For example, the curved section 130 of the delivery needle 120 may include a generally arcuate shape that curls in the lateral direction (e.g., toward the upward direction as shown, for example, in FIG. 5) so as to provide an effective implantation path into the nasal rim 60. In this embodiment, the distal tip portion 122 can penetrate an insertion site 65 in the mucosal surface at the base of the nostril and moved along an implantation path 66 towards the tip 55 of the nose 50 along the nasal rim 60. The practitioner may then drawback the distal tip portion 222 as the implant device 210 is deployed in the nasal rim 60. In another embodiment, the distal tip portion 122 can be inserted at the base of the nostril on the dermal side and moved towards the tip 55 of the nose 50 in a dorsal to ventral path. As such, the curved shape of the section 130 may generally correspond to a portion of the nasal rim curvature, thereby facilitating delivery of the nasal implant device 110 and, in some circumstances, permitting a practitioner to operate the delivery system 100 from an effective position relative to the patient. As described in more detail below, the length of the distal tip portion 122 and the shape of the curved section 130 may be selected by the practitioner depending upon the patient's nasal anatomy (e.g., nose size, rim curvature, tissue thickness, and the like).

Referring to FIG. 2, the nasal implant device 110 may comprise a biocompatible material that is sufficiently flexible to pass through the curved section 130 of the delivery needle 120 (FIG. 1) yet is rigid enough to at least partially support the surrounding nasal tissue. For example, the implant device may comprise Dacron (polyethylene terephthalate), PTFE, silicone, or the like. In this embodiment, the nasal implant device 110 includes a generally flat, thin, and long body and a generally rectangular cross-sectional geometric profile. Such a body shape may serve to stiffen the tissue in the nasal rim. In some embodiments, the nasal implant device 110 may have a length 112 of about 1.0 cm to about 3.8 cm, about 1.5 cm to about 3.5 cm, about 1.8 cm to about 2.8 cm, and about 2.0 cm to about 2.5 cm; may have a thickness 114 of about 0.3 mm to about 3.5 mm, about 0.5 mm to about 3.2 mm, about 1.0 mm to about 3.0 mm, and about 1.2 mm to about 2.0 mm; and may have a width 116 about 0.3 mm to about 3.5 mm, about 0.5 mm to about 3.2 mm, about 1.0 mm to about 3.0 mm, and about 1.2 mm to about 2.0 mm. In this embodiment, the width 116 is greater than the thickness 114, thereby providing the generally rectangular cross-sectional profile. As described in more detail below, the nasal implant device 110 may be trimmed by the practitioner before the implantation procedure so as to customize the length, thickness, or width. In other embodiments, the nasal implant device 110 may comprise a body having a cross-sectional geometric profile that is not rectangular, such as a circular profile, a square profile, an elliptical profile, a hexagonal profile, or the like. In addition, in some embodiments, the nasal implant device 110 may comprise a body having a cross-sectional geometric profile that transitions from a first shape to a second shape, for example, transitions from a rectangular cross-sectional profile to an elliptical cross-sectional profile.

In this embodiment, the nasal implant device 110 may comprise Dacron material that is wound, molded, or otherwise formed to the desired length. For example, implant device 110 may be formed from a braid of Dacron fibers. The Dacron material can be woven into a tight nonresorbable mesh, which allows a slight amount of tissue ingrowth to maintain the implant position in the nasal tissue. In these embodiments, the multifilament arrangement promotes securement between the implant device 110 and the adjacent nasal tissue, which facilitates the position of the implant device 110 immediately after its release from the distal tip portion (FIG. 1). The Dacron material is an example of a polymer material that provides sufficient flexibility to pass through the curved section 130 of the delivery needle 120 and that provides rigidity and resiliency to stiffen the nasal rim 60 (FIG. 1). In addition, the Dacron material may provide a soft feel that causes the implant device 110 to be more discrete when implanted in the nasal tissue (e.g., less detectable by skin palpation or visual inspection of the nose 50).

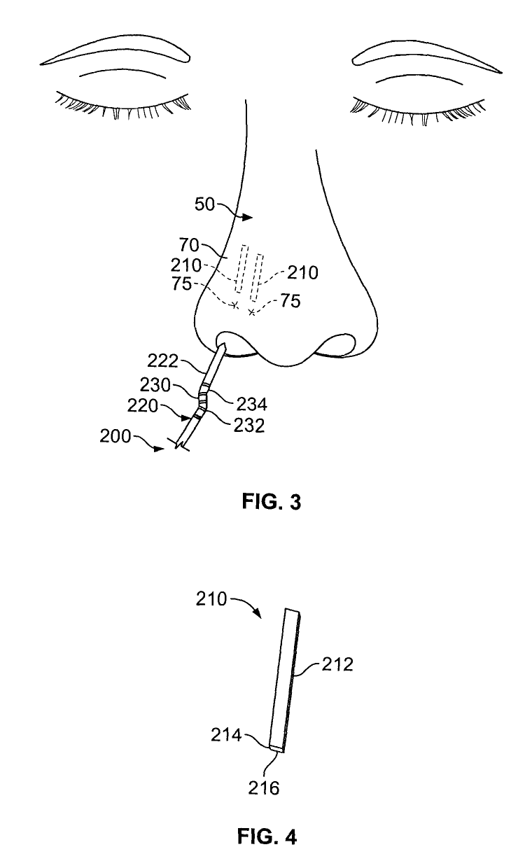

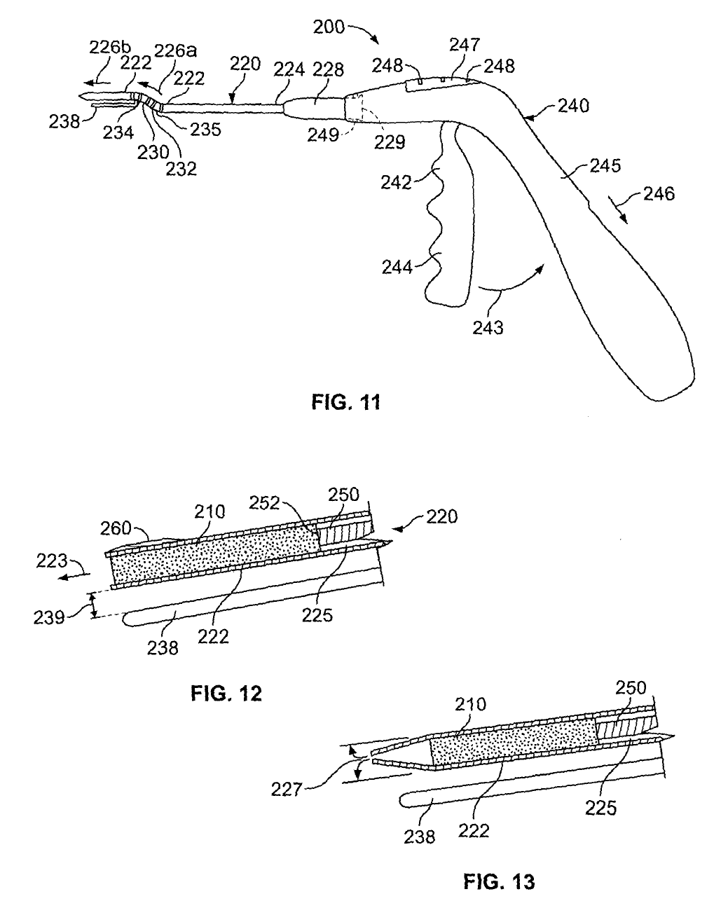

Referring now to FIG. 3, another embodiment of a delivery system 200 may be inserted into nasal tissue proximate to the nasal lateral wall 70 of a nose 50 to position one or more nasal implant devices 210 therein. Similar to the previously described embodiments, the delivery system 200 may include a delivery needle 220 or other cannula device (e.g., a specially configured hypotube, a polymer cannula, or the like) having sufficient rigidity to penetrate into the nasal tissue. The delivery needle 220 includes a distal tip portion 222, a proximal portion 224 (not shown in FIG. 3), and a lumen 225 extending therebetween (not shown in FIG. 3). As described in more detail below in connection with FIG. 11, the delivery system 200 may also include a handle member at a proximal portion coupled with the delivery needle 120 and an actuation member to force the implant device 210 out from the distal tip portion 222.

The distal tip portion 222 of the delivery needle 220 may include a curved section 230 having at least one curve extending at least partially in a lateral direction (e.g., a generally upward direction as shown, for example, in FIG. 11). For example, the curved section 230 of the delivery needle 120 may include a first curve 232 that extends at least partially in the lateral direction and a second curve 234 that extends in a generally longitudinal direction toward the distal tip so as to provide an effective implantation path into the nasal lateral wall 70. Such a curved orientation may facilitate delivery of the implant device 210 into the targeted tissue plane of the nasal lateral wall 70. In this embodiment, the distal tip portion 222 can penetrate an insertion site 75 in the mucosal surface near the end of the nostril and advanced in a cephalo-caudal path into the lateral wall 70. The needle 220 may be inserted into the lateral wall 70 until the distal tip portion 222 reaches the nasal bone (e.g., palpated by the practitioner or visualized on the dermal surface or by tactile feedback when the needle hits the bone). The practitioner may then drawback the distal tip portion 222 as the implant device 210 is deployed. In another embodiment, the distal tip portion 222 can be inserted near the end of the nostril on the dermal side and advanced in a cephalo-caudal path. As such, the curved shape of the curved section 230 may generally correspond to an implantation path in a portion of the nasal lateral wall 70, thereby permitting a practitioner to operate the delivery system 200 from an effective position relative to the patient. As described in more detail below, the length of the distal tip portion 222 and the shape of the curved section 230 may be selected by the practitioner depending upon the patient's nasal anatomy (e.g., nose size, lateral wall length, tissue thickness, and the like).

Referring to FIG. 4, the nasal implant device 210 may comprise a biocompatible material that is sufficiently flexible to pass through the curved section 230 of the delivery needle 220 (FIG. 1) yet is rigid enough to at least partially support the surrounding nasal tissue. For example, the implant device may comprise Dacron (polyethylene terephthalate), PTFE, silicone, or the like. Similar to the previously described embodiments, the nasal implant device 210 includes a generally flat, thin, and long body and a generally rectangular cross-sectional geometric profile. Such a body shape may serve to stiffen the tissue in the nasal lateral wall 70. In some embodiments, the nasal implant device 210 may have a length 212 of about 0.7 cm to about 2.7 cm, about 1.0 cm to about 2.0 cm, and about 1.2 cm to about 1.8 cm; may have a thickness 214 of about 0.2 mm to about 2.5 mm, about 0.5 mm to about 2.0 mm, and about 0.7 mm to about 1.5 mm; and may have a width 216 about 0.2 mm to about 2.5 mm, about 0.5 mm to about 2.0 mm, and about 0.7 mm to about 1.5 mm. In this embodiment, the width 216 is greater than the thickness 214, thereby providing the generally rectangular cross-sectional profile. As previously described, the nasal implant device 210 may comprise a body having a cross-sectional geometric profile that is not rectangular, such as a circular profile, a square profile, an elliptical profile, a hexagonal profile, or the like. In addition, in some embodiments, the nasal implant device 210 may comprise a body having a cross-sectional geometric profile that transitions from a first shape to a second shape, for example, transitions from a rectangular cross-sectional profile to an elliptical cross-sectional profile.

Similar to the previously described embodiments, the nasal implant device 210 may comprise Dacron material that is wound, molded, or otherwise formed to the desired length. For example, implant device 210 may be formed from a braid of Dacron fibers that are woven into a tight nonresorbable mesh. The Dacron material is an example of a polymer material that provides sufficient flexibility to pass through the curved section 230 of the delivery needle 220 and that provides rigidity and resiliency to stiffen the nasal lateral wall (FIG. 3). Also, the Dacron material may have a soft feel that causes the implant device 210 to be more discrete (e.g., less detectable by skin palpation or visual inspection of the nose 50).

Returning now to the embodiments of the delivery system 100 described in connection with FIGS. 1-2, an exemplary embodiment of the delivery system 100 is shown in FIG. 5. As previously described, the delivery needle 120 includes a proximal portion 124 that may be coupled to a handle member 140. In this embodiment, the handle member 140 includes a grasping portion 145 that extends in an at least partially downward direction 146 from the axis of the delivery needle 120 (from the perspective shown in FIG. 5). The curved section 130 of the distal tip portion 122 extends in an at least partially upward direction 126 (from the perspective shown in FIG. 5). As such, the curved section 130 of the distal tip portion 122 extends generally away from the grasping portion 145 of the handle member 140 so that a user may readily grasp the handle member 140 (with the grasping section 145 extending at least partially downward 146 as shown in FIG. 5) while the distal tip portion 122 curves at least partially upward 126 (as shown in FIG. 5). Such a curved orientation of the delivery needle 120 can facilitate delivery of the implant device 110 into the targeted tissue plane of the nasal rim or other nasal anatomy.

Still referring to FIG. 5, the curved section 130 of the delivery needle 120 may include a generally arcuate shape that extends in a lateral direction (e.g., toward the upward direction 126 as shown, for example, in FIG. 5). This arcuate curvature may be selected to substantially correspond to an approximate curvature of at least a portion of the patient's nasal rim 60 (FIG. 1), thereby providing a desirable implantation path into the nasal rim. It should be understood from the description herein that the curved section 130 may include another curvature, such as a transitional curvature that transitions from a first radius of curvature to a second radius of curvature. In some embodiments, the delivery needle 120 may be releasably coupled to the handle member 140 so that a practitioner may select one delivery needle 120 from a plurality of connectable delivery needles 120 (e.g., provided in a kit with the handle member 140) having a variety of curvatures in the curved section 130. Thus, the practitioner may releasably couple the selected delivery needle 120 with the handle member 140 depending upon the nasal anatomy of the patient. For example, a delivery needle 120 having a relatively short radius of curvature may be selected for a patient having a smaller nasal rim circumference. In another example, a delivery needle 120 having a transitional curvature may be selected for a patient having a nasal rim shape that does not correspond to a generally arcuate shape. In addition, the practitioner may be provided with more than one handle member 140 having different sizes (e.g., the kit may include a plurality of delivery needles 120 that are individually connectable with a plurality of handle members 140). In some embodiments, each of the delivery needles 120 in the kit may be preloaded with an implant device 110 (e.g., retained in the needle lumen 125 near the distal tip opening). Alternatively, the user may insert the implant device into the delivery needle 120 before the implantation procedure.

In this embodiment, the delivery needle 120 is releasably coupled with the handle member 140 by an adaptor 128 connected to the needle 120 that mates with a connector 149 of the handle member 140. For example, the adaptor 128 may include a flange portion 129 that can be inserted into a mating condition with a rim connector 149 of the handle member 140. Then, the adaptor may be partially rotated so that the delivery needle locks into position when the curve section 130 extends at least partially in the lateral direction (e.g., the generally upward direction 126 as shown, for example, in FIG. 5). Such a releasably coupling permits the practitioner to select a desirable delivery needle 120 for attachment to the handle member 140.

In some embodiments, the distal tip portion 122 may comprise a malleable material that permits a practitioner to at least partially modify the curved section 130 of the needle 120. For example, the distal tip portion 122 may comprise a ductile aluminum material that can be bent using a hand tool so as to modify the curved section 130. In another example, the distal tip portion 122 may comprise a Beta III titanium alloy (e.g., a titanium alloy including Ti, Mb, Zr, and Sn) that is capable of undergoing plastic flow to take a set shape when subjected to a strain of greater than the set threshold strain. In these examples, the practitioner may select a delivery needle 120 that is suitable for the intended nasal anatomy and may slightly modify the curved section 130 to fine tune the curvature to fit the patient's nasal anatomy.

In some embodiments, the delivery needle 120 may include one or more markings 135 to indicate the depth of tissue penetration during the implantation procedure. For example, the distal tip portion 122 may include a plurality of markings 135 that indicate to a practitioner the depth of needle tip penetration into the nasal rim tissue. Such markings 135 may assist the practitioner in determining the proper deployment position in which the implant device 110 can be arranged. The markings can be positioned on the curved section 130 (as shown in FIG. 5), on the generally noncurved portions of the needle 120, or both.

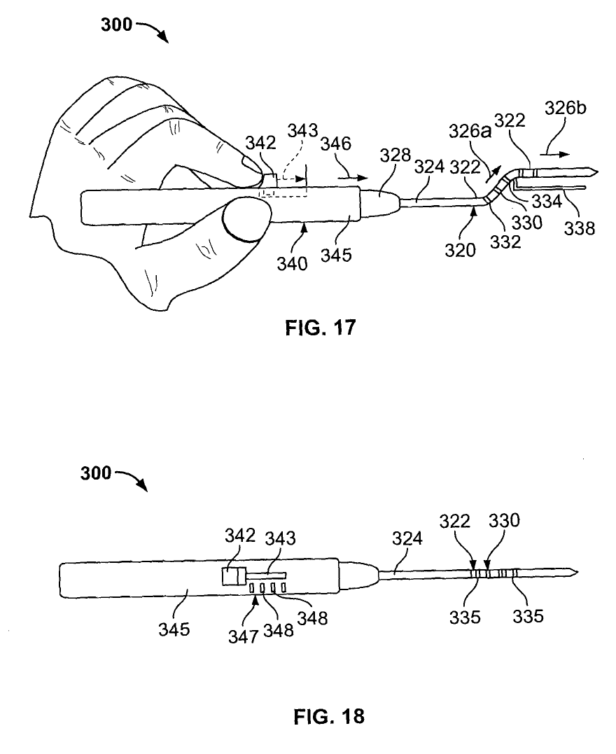

Still referring to FIG. 5, the handle member 140 may include a trigger mechanism 142 to movably adjust the actuation member 150 in the lumen of the delivery needle 120 (described in more detail below in connection with FIGS. 6-7). By applying a force 143 to the trigger mechanism 142, the practitioner may cause the actuation member 150 to apply a deployment force 123 (FIG. 6) to the implant device 110. In this embodiment, the trigger mechanism 142 may include a lever 144 that is pivotably engaged with the grasping section 145 of the handle member 140. As such, the user may hold the grasping section 145 and squeeze the lever 144 towards the grasping section 145, thereby applying a force 143 to deploy the implant device 110. In other embodiments, the trigger mechanism 142 may be a thumb switch along the grasping section 145 that can receive a force from the user's thumb, may be a push rod device extending from the base of the handle member 140, may be an electromechanical device that is activated by a button pressed by the user's thumb or index finger, or the like. In alternative embodiments, the handle member may be grasped like a pencil or the like so that the user's finger can adjust a finger switch (described below in connection with FIGS. 17-18).

As shown in FIG. 5, an actuation indicator 147 may be disposed on the handle member 140. The actuation indicator 147 may inform the user of the length that the actuation member 150 (FIG. 6) has traveled from its nondeployed position. For example, the delivery needle 120 may be inserted in to the nasal tissue while the actuation member 150 is in the nondeployed position (e.g., when the implant device 110 is retained in the lumen 125 of the needle 120 near the distal tip opening). When the delivery needle 120 is positioned in the targeted tissue plane of the nasal rim, the user may apply a force 143 (FIG. 5) to the trigger mechanism 142 that causes the actuation member 150 to deploy the implanted device 110 (FIG. 6). The actuation indicator 147 may display the length of travel of the actuation member 150 in the needle lumen 125 so that the user may know when the implant device 110 is fully deployed. For example, if the implant device 110 has a length 112 (FIG. 2) of 1.8 cm, the user may continue to apply a force 143 to the trigger mechanism 142 and partially withdraw the distal tip portion 122 until the actuation indicator 147 displays a travel length of at least 1.8 cm. Then, the user may fully withdraw the delivery needle 120 from the nasal tissue while the implant device 110 remains deployed in the nasal rim. In this embodiment, the actuation indicator 147 includes one or more markings 148 that indicate the travel length based upon the motion of the trigger mechanism 142 and its interaction with the actuation member 150. In other embodiments, the actuation indicator 148 may include a digital display that shows the travel length based upon one or more sensors disposed in the handle member 140.

Referring to FIG. 6, the actuation member 150 may include at least one surface 152 that engages the implant device 110. In this embodiment, the actuation member 150 may serve as a push rod device that is movably adjusted in response to movement of the trigger mechanism 142 (FIG. 5). As previously described, the user may apply a force 143 to the trigger mechanism 142 that causes the actuation member 150 to travel in a distal direction. When the actuation member 150 travels in the distal direction through the lumen 125, the surface 152 applies a deployment force 123 to the implant device 110.

In some embodiments, the distal tip portion 122 (and possibly the entire delivery needle 120) may include a generally rectangular cross-sectional profile with a generally rectangular lumen 125 extending through the distal tip portion 122. As such, the lumen 125 may receive the implant device 110 having a generally rectangular profile. It should be understood that, in those embodiments in which the implant device has a nonrectangular profile, the distal tip portion 122 and the lumen 125 extending therethrough may also have a non-rectangular profile. Also, in other embodiments, the distal tip portion 122 and the lumen 125 extending therethrough may have a generally circular cross-sectional shape to receive implant devices 110 having a rectangular profile or having different cross-sectional profiles.

Still referring to FIG. 6, the distal tip potion 122 of the delivery needle 120 may include a light source 160 that can be viewed while the distal tip potion 122 is disposed under the dermal surface. For example, the light source 160 may comprise a luminescent coating applied to part of the distal tip portion 122 so that the practitioner can maintain an appropriate tissue depth while advancing the needle 120. The light source 160 may appear brighter to the practitioner as distal tip portion 122 is advanced too close to the dermal surface. In another example, the light source may comprise a miniature LED device disposed on the distal tip portion 122.

Referring to FIG. 7, the distal tip portion 122 may include an adjustable opening 127 that expands when the implant device 110 is deployed from the needle 120. In this embodiment, the distal tip portion 122 may include a distal opening 127 having a closed position (shown in FIG. 7) when the implant device 110 is retained further into the lumen 125. For example, the closed position may provide a more pointed needle tip that facilitates penetration into the nasal tissue. The adjustable opening 127 may be at least partially defined by opposing tip walls that can outwardly flex from the closed position to an open position, as shown by the directional arrows in FIG. 7. As such, when the implant device 110 is forced distally from the needle 120, the opposing tip walls flex outwardly to permit the passage of the implant device 110. In such embodiments, the opposing tip walls that at least partially defined adjustable opening 127 may comprise a biocompatible polymer material or a shape memory material that exhibits superelastic characteristics at or above normal human body temperature (e.g., nitonol material or the like).

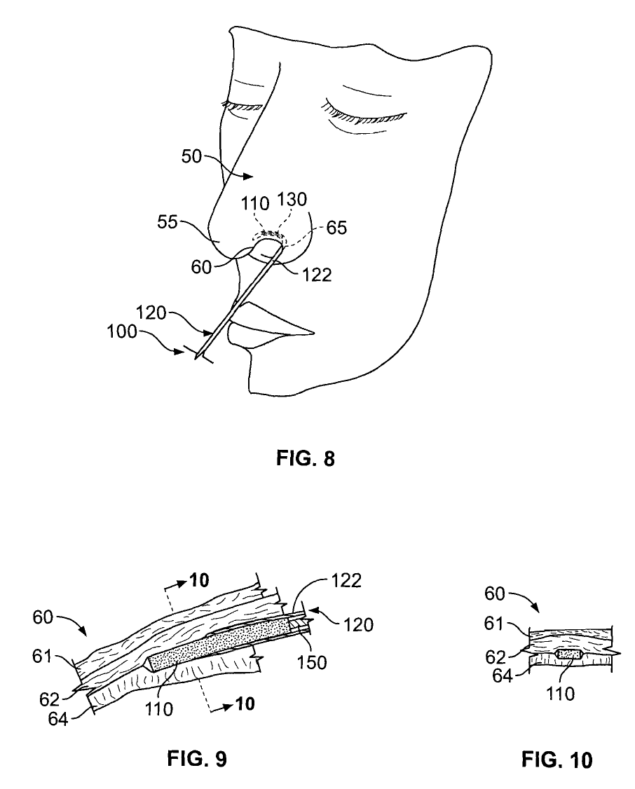

Referring now to FIGS. 8-10, the delivery system 100 may be operated by the practitioner to delivery the implant device 110 to a targeted tissue plane in the nasal rim 60. Implantation into a proper tissue plane can reduce the likelihood of functional failure (e.g., extruding the implant device through the mucosal membrane surface) and cosmetic failure (e.g., the implant device being noticeably viewable on the dermal surface). In the embodiment depicted in FIG. 8, the distal tip portion 122 can penetrate an insertion site 65 in the mucosal surface at the base of the nostril and can be advanced along an implantation path towards the tip 55 of the nose 50 along the nasal rim 60. As shown in FIG. 8, the implant device 110 may be disposed in the lumen of the distal tip portion 122 as the distal tip portion is inserted into the nasal tissue. As previously described, the length of the distal tip portion 122 and the shape of the curved section 130 can be selected by the practitioner depending upon the patient's nasal anatomy (e.g., nose size, rim curvature, tissue thickness, and the like). In this embodiment, the curved shape of the section 130 may substantially correspond to a curved portion of the nasal rim curvature, which can facilitate placement of the implanted device into a targeted tissue plane. Also, the curved shape of the section 130 may permit the practitioner to operate the delivery system 100 from an effective position relative to the patient (e.g., the patient may be resting on his or her back in a reclining chair or on a table surface while the practitioner approaches the patient's nose 50 from a comfortable position in front of the patient or standing over the patient). As previously described, an alternative approach may be employed in which the distal tip portion 122 is inserted at the base of the nostril on the dermal side and moved towards the tip 55 of the nose 50 in a dorsal to ventral path.

As shown in FIGS. 9-10, in some embodiments, the targeted tissue plane in the nasal rim 60 for the implant device 110 may be under the dermal layer 61 and between the SMAS layer 62 (superficial muscle and aponeurotic tissue) and the mucosa layer 64. (It should be understood that the tissue layers 61, 62, and 64 and the implant device 110 are shown for illustrative purposes and are not necessarily shown in scale.) After the distal tip portion 122 has penetrated into the nasal rim and has advanced along the targeted tissue plane, the practitioner may then drawback the distal tip portion 122 while the implant device 110 is deployed in the targeted tissue plane (refer also, for example, to FIG. 1). Accordingly, the delivery system 100 can provide a nonsurgical insertion path the implant device 110 in the targeted tissue plane of the nasal rim 60. The distal tip portion 122 of the delivery needle 120 includes a curved section 130 adapted to penetrate into the targeted tissue plane in the nasal rim and subsequently maintain its position in the targeted tissue plane as it is advanced along the nasal rim 60. In these embodiments, the delivery system 100 can position the implant device 110 in the targeted tissue plane to reduce the likelihood of functional failure and cosmetic failure.

Referring again to the embodiments of the delivery system 200 described in connection with FIGS. 3-4, an exemplary embodiment of the delivery system 200 is shown in FIG. 11. As previously described, the delivery needle 220 includes a proximal portion 224 that may be coupled to a handle member 240. Similar to the previous embodiments, the handle member 240 includes a grasping portion 245 that extends in an at least partially downward direction 246 from the axis of the delivery needle 220 (from the perspective shown in FIG. 11). The curved section 230 of the may include a first curve 232 that extends at least partially in a lateral direction 226a (e.g., a partially upward direction as shown in FIG. 11) and a second curve 234 that extends in a generally longitudinal direction 226b. As such, at least the first curve 232 of the curved section 230 extends generally away from the grasping portion 245 of the handle member 240. Thus, in this embodiment, a user may readily grasp the handle member 240 (with the grasping section 245 extending at least partially downward 246 as shown in FIG. 11) while the distal tip portion 222 curves at least partially upward 226a (as shown in FIG. 11). Such a curved orientation of the delivery needle 220 can facilitate delivery of the implant device 210 into the targeted tissue plane of the nasal lateral wall 70 or other nasal anatomy.

Still referring to FIG. 11, the two curves 232 and 234 of the curved section 230 may provide a distal tip portion 222 that extends longitudinally after a lateral curve, which might be referred to as a “bayonet” shape. This compound curvature may be selected to substantially correspond to an implantation path in a portion of the nasal lateral wall 70, thereby permitting a practitioner to operate the delivery system 200 from an effective position relative to the patient. Also, such a compound curvature may provide the practitioner with a direct line of sight to the implantation site as the distal tip portion 222 approaches the implantation site.

In some embodiments, a guide flange 238 may be coupled to the delivery needle 220 so that the distal tip portion 222 is advanced into the nasal lateral wall 70 at the proper tissue depth. As such, the guide flange 238 may direct the distal tip portion 222 to the targeted tissue plane in the nasal lateral wall 70. Further, the guide flange 238 may reduce the likelihood of the needle tip departing from the targeted tissue plane during advancement (e.g., reduces the likelihood of being advanced too deep or to shallow during insertion). In some embodiments, the guide flange 238 may be fixedly mounted to the delivery needle 220. Alternatively, the guide flange 238 may be releasably engaged with the delivery needle 220 so that the practitioner may elect to use the guide flange 238 in those circumstances where it is most helpful.

Similar to previously described embodiments, the delivery needle 220 may be releasably coupled with the handle member 240 so that a practitioner may select one delivery needle 220 from a plurality of connectable delivery needles 220 (e.g., provided in a kit with the handle member 240) having a variety of curvatures in the curved section 230. Thus, the practitioner may releasably couple a particular delivery needle 220 with the handle member 240 depending upon the nasal anatomy of the patient. In addition, the practitioner may be provided with more than one handle member 240 having different sizes (e.g., the kit may include a plurality of delivery needles 220 that are individually connectable with a plurality of handle members 240). In some embodiments, each of the delivery needles 220 in the kit may be preloaded with an implant device 210 (e.g., retained in the needle lumen 225 near the distal tip opening). Alternatively, the user may insert the implant device into the delivery needle 220 before the implantation procedure. In this embodiment, the adaptor 228 may include a flange portion 229 that can be inserted into a mating condition with a rim connector 249 of the handle member 240. Then, the adaptor 228 may be partially rotated so that the delivery needle 220 locks into position when the curve section 230 extends in the at least partially upward direction 226a (as shown in FIG. 11). Such a releasably coupling permits the practitioner to select a desirable delivery needle 220 for attachment to the handle member 240.

In some embodiments, the distal tip portion 222 may comprise a malleable material that permits a practitioner to at least partially modify the curved section 230 of the needle 220. For example, the distal tip portion 222 may comprise a ductile aluminum material that can be bent using a hand tool so as to modify the curved section 230. In another example, the distal tip portion 222 may comprise a Beta III titanium alloy (e.g., a titanium alloy including Ti, Mb, Zr, and Sn) that is capable of undergoing plastic flow to take a set shape when subjected to a strain of greater than the set threshold strain. In these examples, the practitioner may select a delivery needle 220 that is suitable for the intended nasal anatomy and may slightly modify the curved section 230 to fine tune the curvature to fit the patient's nasal anatomy.

Similar to previously described embodiments, the delivery needle 220 may include one or more markings 235 to indicate the depth of tissue penetration during the implantation procedure. For example, the distal tip portion 222 may include a plurality of markings 235 that indicate to a practitioner the depth of needle tip penetration into the nasal lateral wall tissue. Such markings 235 may assist the practitioner in determining the proper deployment position in which the implant device 210 can be arranged. The markings can be positioned on the curved section 230 (as shown in FIG. 11), on the generally noncurved portions of the needle 220, or both.

Still referring to FIG. 11, similar to previously described embodiments, the handle member 240 may include a trigger mechanism 242 to movably adjust the actuation member 250 in the lumen of the delivery needle 220. By applying a force 243 to the trigger mechanism 242, the practitioner may cause the actuation member 250 to apply a deployment force 223 (FIG. 12) to the implant device 210. In this embodiment, the trigger mechanism 242 includes a lever 244 that is pivotably engaged with the grasping section 245 of the handle member 240. In other embodiments, the trigger mechanism 242 may be a thumb switch along the grasping section 245 that can receive a force from the user's thumb, may be a push rod device extending from the base of the handle member 240, may be an electromechanical device that is activated by a button pressed by the user's thumb or index finger, or the like. In alternative embodiments, the handle member may be grasped like a pencil or the like so that the user's finger can adjust a finger switch (described below in connection with FIGS. 17-18).

Similar to previously described embodiments, an actuation indicator 247 may be disposed on the handle member 240. The actuation indicator 247 may display the length of travel of the actuation member 250 (FIG. 12) in the needle lumen 225 so that the user may know when the implant device 210 is fully deployed. For example, if the implant device 210 has a length 212 (FIG. 4) of 1.2 cm, the user may continue to apply a force 243 to the trigger mechanism 242 and partially withdraw the distal tip portion 222 until the actuation indicator 247 displays a travel length of at least 1.2 cm. Then, the user may fully withdraw the delivery needle 220 from the nasal tissue while the implant device 210 remains deployed in the nasal lateral wall 70. In this embodiment, the actuation indicator 247 includes one or more markings 248 that indicate the travel length based upon the motion of the trigger mechanism 242 and its interaction with the actuation member 250. In other embodiments, the actuation indicator 248 may include a digital display that shows the travel length based upon one or more sensors disposed in the handle member 240.

Referring to FIG. 12, the actuation member 250 may include at least one surface 252 that engages the implant device 210. In this embodiment, the actuation member 250 may serve as a push rod device that is movably adjusted in response to movement of the trigger mechanism 242 (FIG. 11). When the actuation member 250 travels in the distal direction through the lumen 225, the surface 252 applies a deployment force 223 to the implant device 210. Similar to previously described embodiments, the distal tip potion 222 of the delivery needle 220 may include a light source 260 that can be viewed while the distal tip potion 222 is disposed under the dermal surface. For example, the light source 260 may comprise a luminescent coating applied to part of the distal tip portion 222 or may comprise a miniature LED device disposed on the distal tip portion 222.

As previously described, the guide flange 238 may be coupled to the delivery needle 220 so that the distal tip portion 222 is advanced into the nasal lateral wall 70 at the proper tissue depth. The guide flange 238 may be axially spaced apart from the distal tip portion 222 by a clearance space 239 so as to permit a certain amount of nasal tissue therebetween. Accordingly, the guide flange 238 may facilitate advancement of the distal tip portion 222 at the approximate tissue depth to find the targeted tissue plane (refer also to FIGS. 15-16).

Referring to FIG. 13, the distal tip portion 222 may include an adjustable opening 227 that expands when the implant device 210 is deployed from the needle 220, as described in previous embodiments. For example, the distal tip portion 222 may include a distal opening 227 having a closed position (shown in FIG. 13) that provides a more pointed needle tip. When the implant device 210 is forced distally from the needle 220, the opposing tip walls flex outwardly to permit the passage of the implant device 210.

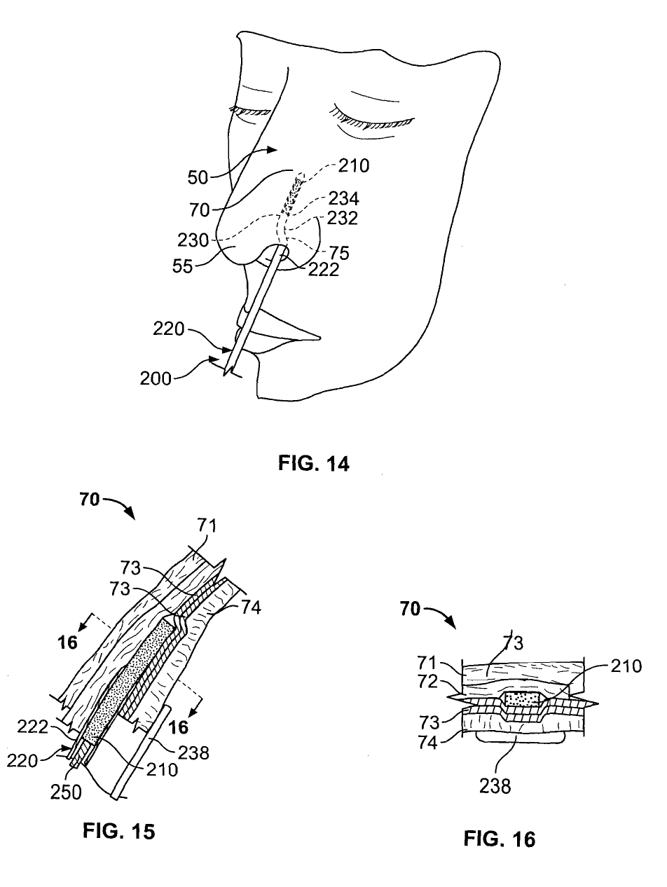

Referring now to FIGS. 14-16, the delivery system 200 may be operated by the practitioner to delivery the implant device 210 to a targeted tissue plane in the nasal lateral wall 70. As previously described, implantation into a proper tissue plane can reduce the likelihood of functional failure (e.g., extruding the implant device through the mucosal membrane surface) and cosmetic failure (e.g., the implant device being noticeably viewable on the dermal surface). In the embodiment depicted in FIG. 14, the distal tip portion 222 can penetrate an insertion site 75 in the mucosal surface near the end of the nostril and can be advanced in a cephalo-caudal path into the lateral wall 70. The needle 220 may be advanced into the lateral wall 70 until the distal tip portion 222 reaches the nasal bone (e.g., palpated by the practitioner or visualized on the dermal surface or by tactile feedback when the needle hits the bone). As shown in FIG. 14, the implant device 210 may be disposed in the lumen of the distal tip portion 222 as the distal tip portion 222 is inserted into the nasal tissue. As previously described, the length of the distal tip portion 222 and the shape of the curved section 230 can be selected by the practitioner depending upon the patient's nasal anatomy (e.g., nose size, tissue thickness, and the like). As such, the curved shape of the section 230 may generally correspond to an implantation path in a portion of the nasal lateral wall 70, thereby permitting a practitioner to operate the delivery system 200 from an effective position relative to the patient (e.g., the patient may be resting on his or her back in a reclining chair or on a table surface while the practitioner approaches the patient's nose 50 from a comfortable position in front of the patient or standing over the patient). As previously described, an alternative approach may be employed in which the distal tip portion 222 in inserted near the end of the nostril on the dermal side and advanced in a cephalo-caudal path.

As shown in FIGS. 15-16, in some embodiments, the targeted tissue plane in the nasal lateral wall 70 for the implant device 210 may be under the dermal layer 71 and between the SMAS layer 72 (superficial muscle and aponeurotic tissue) and the cartilage layers 73, which are above the mucosa layer 74. (It should be understood that the tissue layers 71, 72, 73, and 74 and the implant device 210 are shown for illustrative purposes and are not necessarily shown in scale.) After the distal tip portion 222 has penetrated into the nasal lateral wall and has advanced along the targeted tissue plane, the practitioner may then drawback the distal tip portion 222 while the implant device 210 is deployed in the targeted tissue plane (refer also, for example, to FIG. 3). Accordingly, the delivery system 200 can provide a nonsurgical insertion path the implant device 210 in the targeted tissue plane of the nasal lateral wall 70. The distal tip portion 222 of the delivery needle 220 includes a curved section 230 adapted to penetrate into the targeted tissue plane in the nasal lateral wall 70 and subsequently maintain its position in the targeted tissue plane as it is advanced along the lateral wall 70. In these embodiments, the delivery system 200 can position the implant device 210 in the targeted tissue plane to reduce the likelihood of functional failure and cosmetic failure.

It will be understood that various modifications may be implemented to the delivery system. For example, in some embodiments, a guide flange (similar to flange 238 shown in FIG. 11) may be coupled to the delivery needle 120 (described in connection with FIG. 5). In these embodiments, the guide flange may include an upward curvature similar to the curved section 130. In another example, the delivery system 100 described in connection with FIGS. 4-10 may also be employed to deliver an implant device 210 into the nasal lateral wall 70 (similar to the embodiment shown in FIG. 3). In such embodiments, the distal tip portion 122 (FIG. 5) may penetrate the dermal side of the nasal lateral wall 70 and then be advanced in a cephalo-caudal path into the lateral wall 70.

Furthermore, it should be understood that the embodiments described herein are not limited to the particular examples illustrated in FIGS. 1-16. For example, in some embodiments, a delivery system may have a handle member that is different from those previously illustrated in FIGS. 5 and 11.

Referring to FIGS. 17-18, some embodiments of a delivery system 300 may include a handle member 340 that can be grasped like a pencil or the like so that the user's finger can adjust a finger switch 342. Such a handle member 340 can be engaged primarily by the practitioner's fingers and is capable of providing a high degree of dexterity and control when manipulating the delivery needle 320. The handle member 340 includes a grasping portion 245 that extends in a generally longitudinal direction 346 along the axis of the delivery needle 320 (from the perspective shown in FIGS. 17-18). Similar to previously described embodiments, the delivery needle 320 includes a proximal portion 324 that is coupled to a handle member 340 and a distal tip portion 322 having a curved section 330. The curved section includes a first curve 332 that extends at least partially in a lateral direction 326a (e.g., a partially upward direction as shown in FIG. 17) and a second curve 334 that extends in a generally longitudinal direction 326b. These two curves 332 and 334 of the curved section 330 may provide a distal tip portion 322 that extends longitudinally after a lateral curve, which might be referred to as a “bayonet” shape. This compound curvature may be selected to substantially correspond to an implantation path in a portion of the nasal lateral wall 70, thereby permitting a practitioner to operate the delivery system 300 from an effective position relative to the patient. Also, such a compound curvature may provide the practitioner with a direct line of sight to the implantation site as the distal tip portion 322 approaches the implantation site. Accordingly, in this embodiment, a user may readily grasp the handle member 340 in a manner similar to that of grasping a pencil while the distal tip portion 222 curves at least partially upward 226a (as shown in FIG. 17), which can facilitate delivery of the implant device (e.g., device 210 as described in connection with FIGS. 3-4) into the targeted tissue plane of the nasal lateral wall 70 or other nasal anatomy.

In other embodiments, the delivery system 300 may employ a delivery cannula other than the “bayonet” shape delivery needle 320. For example, in some embodiments, the delivery system 300 may include a delivery needle have a distal tip portion similar to that of the delivery needle 120 described in connection with FIGS. 5-10. In such circumstances, the delivery needle configured to delivery a nasal implant into the nasal rim is coupled to the handle member 340. Thus, a user may readily grasp the handle member 340 in a manner similar to that of grasping a pencil while the distal tip portion curls at least partially in a lateral direction to facilitate the implant delivery to the nasal rim (refer, for example, to FIG. 8).

Similar to the embodiments previously described in connection with FIGS. 1-16, the delivery needle 320 may be coupled with a guide flange 338 so that the distal tip portion 322 is advanced into the nasal lateral wall 70 at the proper tissue depth. The distal tip portion 322 may include an adjustable opening that expands when the implant device (e.g., implant device 210) is deployed from the needle 320 (described in connection with FIGS. 6-7 and 12-13). Also, similar to previously described embodiments, the delivery needle 320 may be releasably coupled with the handle member 340 so that a practitioner may select one delivery needle 320 from a plurality of connectable delivery needles 320 (e.g., provided in a kit with the handle member 340) having a variety of curvatures in the curved section 330. Furthermore, in some embodiments, the distal tip portion 322 may comprise a malleable material that permits a practitioner to at least partially modify the curved section 330 of the needle 320 (described in connection with FIG. 11). Similar to previously described embodiments, the delivery needle 320 may include one or more markings 335 (FIG. 8) to indicate the depth of tissue penetration during the implantation procedure. Finally, similar to previously described embodiments, the distal tip potion 322 of the delivery needle 320 may include a light source that can be viewed while the distal tip potion 322 is disposed under the dermal surface.

Still referring to FIGS. 17-18, the handle member 340 includes a trigger mechanism 342 to movably adjust the actuation member in the lumen of the delivery needle 320. By applying a force 243 to the trigger mechanism 242 (as shown in FIG. 17), the practitioner may cause the actuation member to apply a deployment force to the implant device. In this embodiment, the trigger mechanism 342 includes a finger-actuated switch that is slidable within a guide slot 343 (FIG. 18) of the handle member 340. Similar to previously described embodiments, an actuation indicator 347 may be disposed on the handle member 340. The actuation indicator 347 may display the length of travel of the actuation member in the needle lumen so that the user may know when the implant device is fully deployed. In this embodiment, the actuation indicator 347 includes one or more markings 348 that indicate the travel length based upon the motion of the trigger mechanism 342 and its interaction with the actuation member. As previously described in connection with FIGS. 14-16, the delivery system 300 may be operated by the practitioner to delivery the implant device (e.g., implant device 210) to a targeted tissue plane in the nasal lateral wall 70. Implantation into a proper tissue plane can reduce the likelihood of functional failure (e.g., extruding the implant device through the mucosal membrane surface) and cosmetic failure (e.g., the implant device being noticeably viewable on the dermal surface).

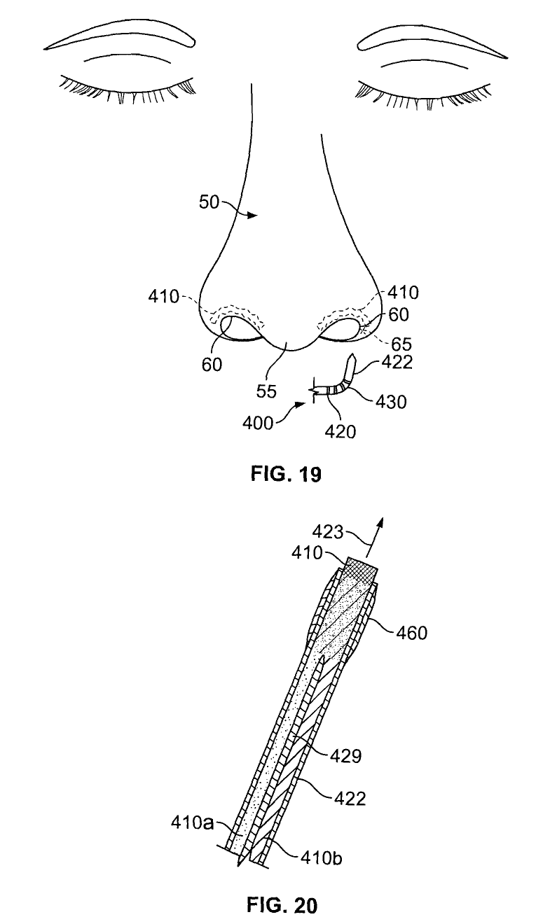

Referring now to FIGS. 19-20, some embodiments of a delivery system 400 may be configured to deliver a nasal implant material in a non-rigid state, which stiffens after to a more rigid or resilient state after deployment to the nasal tissue. As shown in FIG. 19, the delivery system 400 may be inserted into nasal tissue proximate to the nostril rim 60 of a nose 50 in a manner similar to the embodiments previously described in connection with FIGS. 1 and 8-10. The delivery system 400 includes a delivery needle 420 or other cannula device (e.g., a specially configured hypotube, a polymer cannula, or the like) having sufficient rigidity to penetrate into the nasal tissue. The delivery needle 420 includes a distal tip portion 422, a proximal portion (not shown in FIG. 19), and one or more lumens 425 extending therebetween (refer to FIG. 20). Similar to previously described embodiments, the delivery system 400 may also include a handle member at a proximal portion coupled with the delivery needle 420 (refer, for example, to FIGS. 5, 11, and 17-18).

Also, as previously described in connection with FIGS. 1 and 8-10, the distal tip portion 422 of the delivery needle 420 may include a curved section 430 extending at least partially in a lateral direction. Such a curved orientation may facilitate delivery of the implant 410 into the targeted tissue plane of the nasal rim 60. For example, the curved section 430 of the delivery needle 420 may include a generally arcuate shape that curls in the lateral direction so as to provide an effective implantation path into the nasal rim 60. In this embodiment, the distal tip portion 422 can penetrate an insertion site 65 in the mucosal surface at the base of the nostril and moved along an implantation path towards the tip 55 of the nose 50 along the nasal rim 60. The practitioner may then drawback the distal tip portion 422 as the implant 410 is deployed in the nasal rim 60. The length of the distal tip portion 422 and the shape of the curved section 430 may be selected by the practitioner depending upon the patient's nasal anatomy (e.g., nose size, rim curvature, tissue thickness, and the like).

In use, the one or more lumens of the delivery needle 420 may be in fluid communication with one or more fluid reservoirs arranged in the handle member (not shown in FIG. 19). For example, a syringe-type reservoir may be arranged in the handle member so that a trigger mechanism can be actuated to forwardly advance a plunger of the syringe-type reservoir. Thus, the reservoir can be used to advance at least a first agent 410a through the delivery needle 420 (described below), which also provides the actuation force 423 to deploy the nasal implant 410 from the delivery needle 420.

Referring to FIG. 20, the nasal implant device 410 may comprise one or more biocompatible materials that are advanced through the delivery needle 422 in a generally non-rigid state and thereafter stiffen to form a resilient implant 410 that is rigid enough to at least partially support the surrounding nasal tissue. For example, the implant 410 may comprise a first agent 410 that is advanced through a first lumen of the delivery needle 422 toward the distal port. The first agent 410a may comprise a material that reacts with a second agent 410b to form a resilient material for the deployed implant 410.

For example, the first agent 410a may comprise a polymer resin in a non-rigid, fluid state that can be combined with a second polymer resin 410b (passing through a second lumen of the delivery needle 420) during deployment. When the first and second resins 410a and 410b are combined, the resulting material polymerizes or sets into a more rigid and resilient product. This resulting material can be forced from the delivery needle 420 by an actuation force 423 and into the targeted nasal tissue where it serves as the nasal implant 410.

In another example, the first agent 410a may comprise a polymer material in a non-rigid state (e.g., heated to an elevated temperature) that reacts with or is cooled by a second agent 410b, such as air, water, or saline, during deployment. The polymer material 410a can be fully or partially stiffening as it is deployed from the port of the delivery needle 420. As such, the polymer material may form a resilient nasal implant 410 that is deployed into the targeted tissue plane.

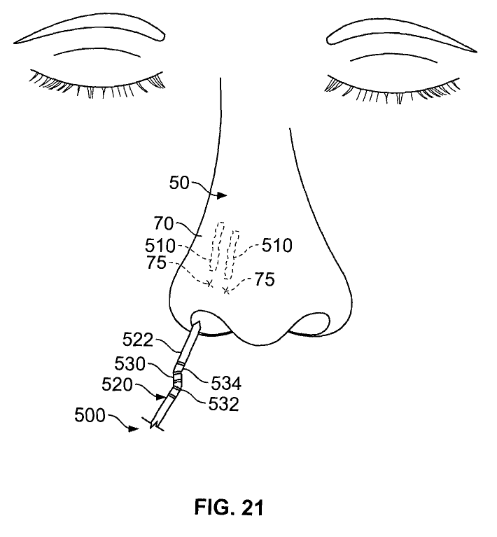

In other embodiments, the delivery system may employ a delivery cannula other than the arcuate curved delivery needle 420. For example, as shown in FIG. 21, the delivery system 500 may include a delivery needle 520 having a distal tip portion 522 capable of delivering one or more nasal implants to the nasal lateral walls. Similar to the embodiments described in connection with FIGS. 19-20, the delivery system 500 is configured to deliver a nasal implant material in a non-rigid state, which stiffens after to a more rigid or resilient state after deployment to the nasal tissue the delivery needle includes one or more lumens.

The distal tip portion 522 of the delivery needle 520 may include a curved section 530 having at least one curve extending at least partially in a lateral direction. For example, the curved section 530 of the delivery needle 520 may include a first curve 532 that extends at least partially in the lateral direction and a second curve 534 that extends in a generally longitudinal direction toward the distal tip so as to provide an effective implantation path into the nasal lateral wall 70. Such a curved orientation may facilitate delivery of the nasal implant 510 into the targeted tissue plane of the nasal lateral wall 70. In this embodiment, the distal tip portion 522 can penetrate an insertion site 75 in the mucosal surface near the end of the nostril and advanced in a cephalo-caudal path into the lateral wall 70. The needle 520 may be inserted into the lateral wall 70 until the distal tip portion 522 reaches the nasal bone (e.g., palpated by the practitioner or visualized on the dermal surface or by tactile feedback when the needle hits the bone). The practitioner may then drawback the distal tip portion 522 as the implant device 210 is deployed. In another embodiment, the distal tip portion 522 can be inserted near the end of the nostril on the dermal side and advanced in a cephalo-caudal path. As such, the curved shape of the curved section 530 may generally correspond to an implantation path in a portion of the nasal lateral wall 70, thereby permitting a practitioner to operate the delivery system 500 from an effective position relative to the patient. As previously described, the length of the distal tip portion 522 and the shape of the curved section 530 may be selected by the practitioner depending upon the patient's nasal anatomy (e.g., nose size, lateral wall length, tissue thickness, and the like).

In use, the one or more lumens of the delivery needle 520 may be in fluid communication with one or more fluid reservoirs arranged in the handle member (not shown in FIG. 21). In one example, a syringe-type reservoir may be arranged in the handle member so that a trigger mechanism can be actuated to forwardly advance a plunger of the syringe-type reservoir. Accordingly, the reservoir can be used to advance at least a first agent through the delivery needle 520, which also provides the actuation force to deploy the nasal implant 510 from the delivery needle 520. Similar to the embodiments previously describe din connection with FIG. 20, the nasal implant 510 may comprise one or more biocompatible materials that are advanced through the delivery needle 522 in a generally non-rigid state and thereafter stiffen to form a resilient implant 510 that is rigid enough to at least partially support the surrounding nasal tissue. For example, the first agent may comprise a material that reacts with a second agent to form a resilient material for the deployed implant 410.

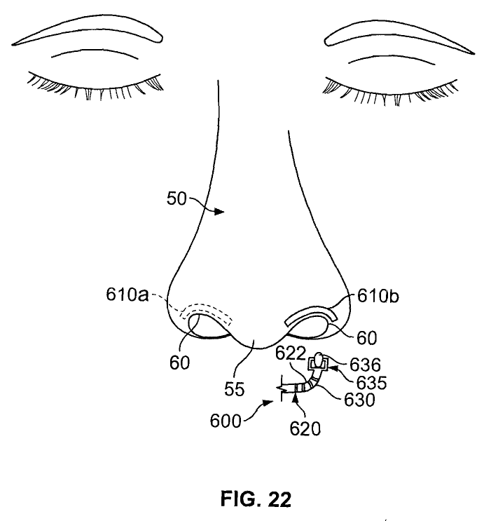

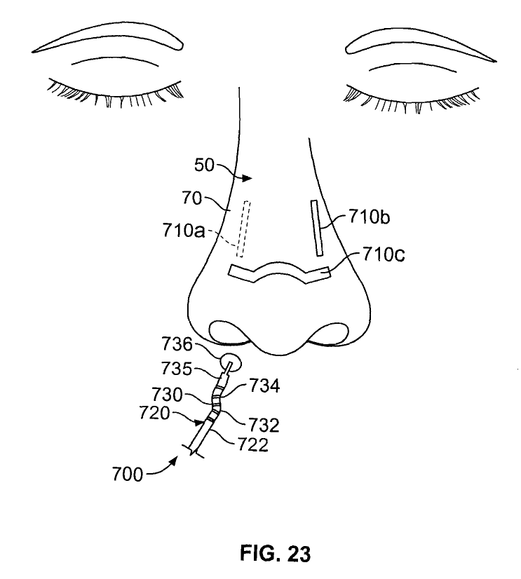

Referring now to FIGS. 22-23, some embodiments of a delivery system 600 or 700 may include a delivery instrument 620 or 720 configured to apply a rigidity supplement material to a targeted region of nasal tissue. As shown in FIG. 22, the delivery system 600 may be adapted to apply the rigidity supplement material to an internal portion or external portion of the nasal rim 60. As shown in FIG. 23, the delivery system 700 may be adapted to apply the rigidity supplement material to an internal portion or external portion of the lateral nasal wall 70 or across the bridge of the nose along the lateral nasal walls 70.

In some embodiments, the rigidity supplement material may comprise a substantially translucent or transparent polymer that is applied in a non-rigid, generally liquid state. The polymer material is selected to be biocompatible with the outer dermal layers and the mucous membranes. In this embodiment, the polymer can provide elastic properties after it has transitioned from the non-rigid state to a resilient state. For example, after the polymer material is applied to the nasal skin or the mucous membranes, it may partially dry and thereafter contract to provide a stiffening effect on the nasal structure to which it was applied. In some embodiments, the polymer may react via air after being applied to the targeted nasal tissue. Alternatively, the polymer may be applied as two or more resins that react and set to form a resilient product when applied to the nasal tissue. After a particular duration of use, the polymer material may be removed from the nasal tissue either by peeling away from the tissue or by washing with a solvent. Accordingly, the delivery systems 600 and 700 can provide a non-surgical treatment option that can be comfortably used during both day and night. In particular, the rigidity supplement material can be applied to either the internal or external portions of the nose 50 without being noticeably visible on the dermal surface.

Referring to FIG. 22, the delivery system 600 includes a delivery instrument 620 having sufficient rigidity to press an applicator 635 to the targeted tissue. The delivery instrument 620 includes a distal tip portion 622 and a proximal portion (not shown in FIG. 22). Similar to previously described embodiments, the delivery system 600 may also include a handle member at a proximal portion coupled with the delivery instrument 620 (refer, for example, to FIGS. 5, 11, and 17-18). Also, as previously described in connection with FIGS. 1, 5 and 8, the distal tip portion 622 of the delivery instrument 620 may include a curved section 630 extending at least partially in a lateral direction. Such a curved orientation may facilitate application of the rigidity supplement material 610a onto the internal portion of the nasal rim 60. For example, the curved section 630 of the delivery instrument 620 may include a generally arcuate shape that curls in the lateral direction so as to provide an effective application path to the nasal rim 60. In addition, such a curved orientation may facilitate application of the rigidity supplement material 610b onto the external portion of the nasal rim 60. The length of the distal tip portion 622 and the shape of the curved section 630 may be selected by the practitioner depending upon the patient's nasal anatomy (e.g., nose size, rim curvature, tissue thickness, and the like).

The delivery instrument 620 includes an applicator 635 that contacts the targeted nasal tissue and deposits one or more layers of the rigidity supplement material 610a and 610b. In this embodiment, the applicator 635 comprises a miniature roller 636 that receives the previously described polymer material in the non-rigid, generally liquid state. In other embodiments, the applicator 635 may comprise a miniature brush having bristles that receive the polymer material in the non-rigid, generally liquid state. The delivery instrument 620 can be manipulated by the user (e.g., using the handle member) so that the applicator 635 is brought into contact with the targeted nasal tissue and the polymer material is deposited thereon. Thereafter, the polymer can partially dry or otherwise stiffen to provide supplemental rigidity to the nasal structure to which it was applied.

Referring to FIG. 23, the delivery system 700 includes a delivery instrument 720 having sufficient rigidity to press an applicator 735 to the targeted tissue. The delivery instrument 720 includes a distal tip portion 722 and a proximal portion (not shown in FIG. 23). Similar to previously described embodiments, the delivery system 700 may also include a handle member at a proximal portion coupled with the delivery instrument 720 (refer, for example, to FIGS. 5, 11, and 17-18). Also, as previously described in connection with FIGS. 3, 11, and 14, the distal tip portion 722 of the delivery instrument 720 may include a curved section 730 extending at least partially in a lateral direction. For example, the curved section 730 of the delivery instrument 720 may include a first curve 732 that extends at least partially in the lateral direction and a second curve 734 that extends in a generally longitudinal direction toward the distal tip so as to provide an effective application path to the nasal lateral wall 70. Such a curved orientation may facilitate application of the rigidity supplement material 710a onto the internal portion of the nasal lateral wall 70. In addition, such a curved orientation may facilitate application of the rigidity supplement material 710b onto the external portion of the nasal lateral wall 70. The delivery instrument 720 may also be used to apply the rigidity supplement material 710c onto an external portion of the nasal bridge along both nasal lateral walls 70. The length of the distal tip portion 722 and the shape of the curved section 730 may be selected by the practitioner depending upon the patient's nasal anatomy (e.g., nose size, rim curvature, tissue thickness, and the like).

The delivery instrument 720 includes an applicator 735 that contacts the targeted nasal tissue and deposits one or more layers of the rigidity supplement material 710a, 710b, and 710c. Similar to the embodiments previously described in connection with FIG. 22, the applicator 735 comprises a miniature roller 736 that receives the previously described polymer material in the non-rigid, generally liquid state. In other embodiments, the applicator 735 may comprise a miniature brush having bristles that receive the polymer material in the non-rigid, generally liquid state. The delivery instrument7 can be manipulated by the user (e.g., using the handle member) so that the applicator 735 is brought into contact with the targeted nasal tissue and the polymer material is deposited thereon. Thereafter, the polymer can partially dry or otherwise stiffen to provide supplemental rigidity to the nasal structure to which it was applied.

A number of embodiments of the invention have been described. Nevertheless, it will be understood that various modifications may be made without departing from the spirit and scope of the invention. Accordingly, other embodiments are within the scope of the following claims.

What is claimed is:

1. A nasal implant delivery system comprising:
a delivery cannula including a distal portion, a proximal portion, and a lumen extending therebetween, at least a section of the proximal portion extending in longitudinal direction along a central axis, the distal portion of the delivery cannula including a curved section that extends in an at least partially lateral direction away from the central axis and including a port to deliver a nasal implant;
an actuation member movable disposed in the lumen, the actuation member including a distal surface to at least partially engage the nasal implant when the implant is disposed in the lumen; and
a handle member coupled with the delivery cannula to manipulate the position of the distal portion relative to a targeted site, the handle member being coupled to a hand-adjustable switch to cause the actuation member to move in the lumen.

2. The nasal implant delivery system of claim 1, wherein the curved section of the distal portion of the delivery cannula is penetrable into a tissue plane of a nasal rim.

3. The nasal implant delivery system of claim 2, wherein the curved section of the delivery cannula curls in a generally arcuate shape away from the central axis.

4. The nasal implant delivery system of claim 3, wherein the generally arcuate shape of the curved section is substantially congruent to an anatomical curvature to at least a portion of a nasal rim.

5. The nasal implant delivery system of claim 1, wherein the curved section of the distal portion of the delivery cannula is penetrable into a tissue plane of a lateral nasal wall.

6. The nasal implant delivery system of claim 5, wherein the curved section of the delivery cannula includes a first curve that extends in the at least partially lateral direction away from the central axis and a second curve that extends in a longitudinal direction generally parallel to the central axis toward a distal tip of the delivery cannula.

7. The nasal implant delivery system of claim 6, wherein the distal tip is insertable into a mucosal surface and advanceable in a cephalo-caudal direction toward the targeted tissue plane.

8. The nasal implant delivery system of claim 1, further comprising a guide flange offset from and extending generally parallel to at least a part of the distal portion of the delivery cannula.

9. The nasal implant delivery system of claim 1, wherein at least the distal portion of the delivery cannula comprises a needle.

10. The nasal implant delivery system of claim 1, further comprising a nasal implant arranged in the distal portion of the delivery cannula.

11. The nasal implant delivery system of claim 10, wherein the nasal implant comprises a resilient elongate structure that is deliverable to a targeted nasal tissue plane.

12. A nasal implant delivery system, comprising:
a delivery cannula including a distal portion, a proximal portion, and at least one lumen extending therebetween, at least a section of the proximal portion extending in longitudinal direction along a central axis, the distal portion of the delivery cannula including a curved section that extends in an at least partially lateral direction away from the central axis and including a port in communication with the at least one lumen to deploy a nasal implant material;
a fluid reservoir in fluid communication with the at least one lumen, the fluid reservoir containing a polymer material in a generally non-rigid state; and
an actuation member to deploy the polymer material from the distal portion of the delivery cannula and to a targeted nasal tissue plane, the polymer material transitioning to a resilient state when deployed from the distal portion.

13. The nasal implant delivery system of claim 12, wherein the curved section of the distal portion of the delivery cannula is penetrable into a tissue plane of a nasal rim.

14. The nasal implant delivery system of claim 13, wherein the curved section of the delivery cannula curls in a generally arcuate shape away from the central axis.

15. The nasal implant delivery system of claim 14, wherein the generally arcuate shape of the curved section is substantially congruent to an anatomical curvature to at least a portion of a nasal rim.

16. The nasal implant delivery system of claim 12, wherein the curved section of the distal portion of the delivery cannula is penetrable into a tissue plane of a lateral nasal wall.

17. The nasal implant delivery system of claim 16, wherein the curved section of the delivery cannula includes a first curve that extends in the at least partially lateral direction away from the central axis and a second curve that extends in a longitudinal direction generally parallel to the central axis toward a distal tip of the delivery cannula.

18. The nasal implant delivery system of claim 17, wherein the distal tip is insertable into a mucosal surface and advanceable in a cephalo-caudal direction toward the targeted tissue plane.

19. The nasal implant delivery system of claim 12, further comprising a guide flange offset from and extending generally parallel to at least a part of the distal portion of the delivery cannula.

20. The nasal implant delivery system of claim 12, wherein at least the distal portion of the delivery cannula comprises a needle.

21. A delivery system for a nasal rigidity supplement, comprising:
a delivery instrument including a distal portion and a proximal portion, the proximal portion extending in longitudinal direction along a central axis, the distal portion of the delivery instrument including an applicator to contact a targeted nasal tissue site; and
a rigidity supplement material arranged on the applicator of the delivery instrument, the rigidity supplement material being in a generally non-rigid state when arranged on the applicator,
wherein the rigidity supplement material transitions to a resilient state when deployed from the applicator to the targeted nasal tissue site.

22. The delivery system of claim 21, wherein the rigidity supplement material reacts with air to transition to the resilient state.

23. The delivery system of claim 21, wherein the rigidity supplement material comprises a substantially translucent or transparent material transitions when in the resilient state.

24. The delivery system of claim 21, wherein the applicator comprises a brush device.

25. The delivery system of claim 21, wherein the applicator comprises a roller device.

26. The delivery system of claim 21, the rigidity supplement material comprises a generally elastic laminate material transitions when in the resilient state.
